(19) United States
(12) Patent Application Publication
(10) Pub. No.: US 2012/0016417 A1
(43) Pub. Date: Jan. 19, 2012

(54) FLEXING LINKS FOR INTERVERTEBRAL STABILIZATION

(75) Inventor: Calin Druma, Cupertino, CA (US)

(73) Assignee: KYPHON SARL, Neuchatel (CH)

(21) Appl. No.: 12/836,782

(22) Filed: Jul. 15, 2010

(51) Int. Cl. A61B 17/70

(52) U.S. Cl. 606/246; 606/278; 606/279

(57) ABSTRACT

A flexing link assembly and methods for implanting such an assembly in a patient are disclosed herein. The flexing link assembly includes a pair of links that are movably connected together adjacent to an intermediate portion of each link. The connection between the links allows relative movement between the links. One end of each link includes an attachment portion adapted to facilitate attachment of the links to an inferior vertebra. The other end of each link is adapted to facilitate engagement with a spinous process of an adjacent superior vertebra. This arrangement of the links allows flexing and relative movement of adjacent vertebrae yet limits extension therebetween.

### BACKGROUND

This invention relates generally to the treatment of spinal conditions, and more particularly, to the treatment of spinal stenosis using devices for implantation between adjacent spinous processes.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis. The principal goal of surgery is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia as an incision is made in the patient to access the spine. The lamina of one or more vertebrae is removed to create more space for the nerves. The intervertebral disc may also be removed, and the adjacent vertebrae may be fused to strengthen the unstable segments. The success rate for decompressive laminectomy has been reported to be in excess of 65%. A significant reduction of the symptoms of lumbar spinal stenosis is also achieved in many of these cases.

Alternatively, the vertebrae can be distracted and an interspinous process device implanted between adjacent spinous processes of the vertebrae to maintain the desired separation between the vertebral segments. Such interspinous process devices typically work for their intended purposes. However, because of the human anatomy some of these devices are not readily usable between certain vertebrae.

The spine is divided into regions that include the cervical, thoracic, lumbar, and sacrococcygeal regions. The cervical region includes the top seven vertebrae indentified as C1-C7. The thoracic region includes the next twelve vertebrae identified as T1-T12. The lumbar region includes five vertebrae L1-L5. The sacrococcygeal region includes nine fused vertebrae that make up the sacrum and the coccyx. The vertebrae of the sacrum are identified as the S1-S5 vertebrae. Four or five rudimentary members form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebrae angled from about 30 degrees to about 60 degrees in the horizontal plane. The S1 vertebra includes a spinous process aligned along a ridge called the medial sacral crest. However, the spinous process on the S1 vertebra may not be well defined, or may be non-existent, and therefore may not be adequate for supporting an interspinous process device positioned between the L5 and S1 spinous processes.

Thus, a need exists for a mechanism that will allow an interspinous process device to be readily positioned between the L5 and S1 spinous processes so that the L5 and S1 vertebrae can be distracted and the interspinous process device can maintain the desired separation between the vertebral segments.

### SUMMARY OF THE INVENTION

A flexing link assembly is described herein that includes two links that are attached to the sacrum and connected together in such a manner to engage the spinous process of the L5 vertebra. Each link includes an attachment portion adapted to be attached to the sacrum, an intermediate portion extending from the attachment portion and adapted to be connected to the other link and a mounting portion extending from the intermediate portion and adapted to facilitate engagement with the spinous process of the L5 vertebra. Any fixation device may be used to attach each link to the sacrum. For example, a standard facet or pedicle screw may be used for this purpose. The links may be connected to each other by any suitable means, such as a mating screw. If desired, the surfaces of the links adjacent to the location where they are connected together may be ground in a manner that facilitates mating and engagement between the two surfaces. For example, complementary grooved configurations may be etched into the mating surfaces of the intermediate portion of each link. The connection between the two links is configured to allow relative motion between the two links. In this way, the connection between the two links allows the flexing link assembly to flex and thus allows relative motion between the L5 vertebra and the sacrum. Even with this flexing, the flexing link assembly still provides decompression between the L5 vertebra and the sacrum. Any fixation device may be used to attach each link to the spinous process of the L5 vertebra. For example, spikes, screws or tethers may be used. Alternatively, the spinous process of the L5 vertebra does not have to be rigidly attached to the mounting portions of each link. Instead, the flexing link assembly may be configured to limit extension yet still allow flexion, lateral bending and rotation of the spine.

Each link may be implanted in the patient so that the attachment portions are located on opposite sides of the medial sacral crest. The configuration of each link allows the intermediate portion to extend medially from the attachment portion. This will allow the intermediate portion to cross the medial sacral crest. The mounting portion extends in a generally cephalic direction from the intermediate portion. This allows the mounting portion to be located in close proximity to the spinous process of the immediately superior vertebra. The configuration of each link positions the mounting portion on the opposite side of the sagittal plane from the attachment portion. With the attachment portion of one link located on one side of the medial sacral crest and the attachment portion of the other link located on the opposite side of the medial sacral crest, the two links cross each other adjacent to their intermediate portions at a point generally aligned with the sagittal plane. In this orientation, both mounting portions are adjacent to the L5 spinous process. The mounting portions can thus be attached to the L5 spinous process so as to maintain decompression between the S1 and the L5 vertebra. Alternatively, as mentioned above, the mounting portions do not have to be attached to the spinous process of the L5 vertebra. Instead they may together have a configuration that engages the inferior surface of the spinous process of the L5 vertebra to maintain decompression. This allows flexion between the two vertebrae and still limits extension. This configuration would also allow lateral bending and rotation of the spine.

### BRIEF DESCRIPTION OF THE DRAWINGS

FIG. 1 is a perspective view of one embodiment of a flexing link assembly;

FIG. 2 is an exploded perspective view of the flexing link assembly shown in FIG. 1;

FIG. 3 is a perspective view of the flexing link assembly shown in FIG. 1 mounted to the sacrum of a patient;

FIG. 4 is an elevation view of the flexing link assembly shown in FIG. 1 mounted to the sacrum of a patient;

FIG. 5 is a cross-sectional view taken along line 5-5 of the flexing link assembly shown in FIG. 1;

FIG. 6 is a cross-sectional view taken along line 6-6 of one of the links of the flexing link assembly shown in FIG. 1;

FIG. 7 is a perspective view of another embodiment of a flexing link assembly;

FIG. 8 is an exploded perspective view of the flexing link assembly shown in FIG. 7;

FIG. 9 is a perspective view of the flexing link assembly shown in FIG. 7 mounted to the sacrum of a patient;

FIG. 10 is an elevation view of the flexing link assembly shown in FIG. 7 mounted to the sacrum of a patient;

FIG. 11 is a perspective view of another embodiment of a flexing link assembly;

FIG. 12 is an exploded view of the flexing link assembly shown in FIG. 11;

FIG. 13 is a cross-sectional view taken along line 13-13 of the flexing link assembly shown in FIG. 11;

FIG. 14 is a perspective view of one half of the ball joint used in the flexing link assembly shown in FIG. 11;

FIG. 15 is a perspective view of the other half of the ball joint used in the flexing link assembly shown in FIG. 11;

FIG. 16 is a perspective view of the flexing link assembly shown in FIG. 11 mounted to the sacrum of a patient;

FIG. 17 is an elevation view of the flexing link assembly shown in FIG. 11 mounted to the sacrum of a patient; and

FIG. 18 is an elevation view of yet another embodiment of a flexing link assembly mounted to the sacrum of a patient.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

As used in this specification and the appended claims, the term “body” when used in connection with the location where the device of this invention is to be placed to treat lumbar spinal stenosis, or to teach or practice implantation methods for the device, means a mammalian body or a model of a mammalian body. For example, a body can be a patient's body, or a cadaver, or a portion of a patient's body or a portion of a cadaver or a model of any of the foregoing.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

A flexing link assembly 10 is described herein that includes two links, a left link 100, and a right link 200, that are attached to a vertebra, typically the sacrum, and connected together in such a manner to engage the spinous process of the immediately superior vertebra, typically the L5 vertebra. Although, flexing link assembly 10 is described herein has being attached at a lower end to the sacrum and attached at an upper end to the spinous process of the L5 vertebra, it is to be understood that flexing link assembly 10 could be connected to other adjacent vertebrae. In addition, it is to be understood that flexing link assembly 10 does not have to be attached to the adjacent superior vertebra. Instead, it may be attached to an inferior vertebra and be adapted to engage, but not be attached to, the adjacent superior vertebra. In the embodiments where flexing link assembly 10 is not attached to the adjacent superior vertebra, left link 100 and right link 200 have a configuration so that they contact the inferior surface of the adjacent superior vertebra to thus maintain the desired distraction between the adjacent vertebrae.

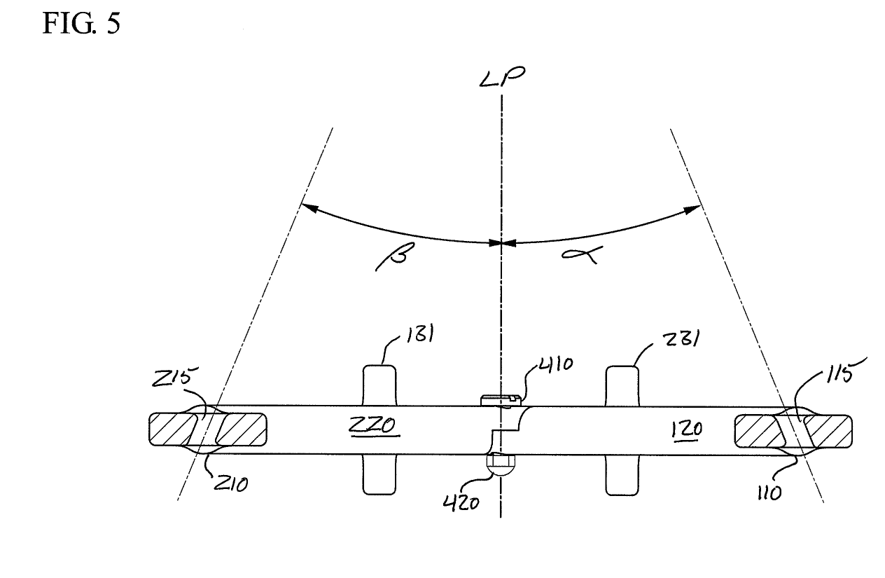

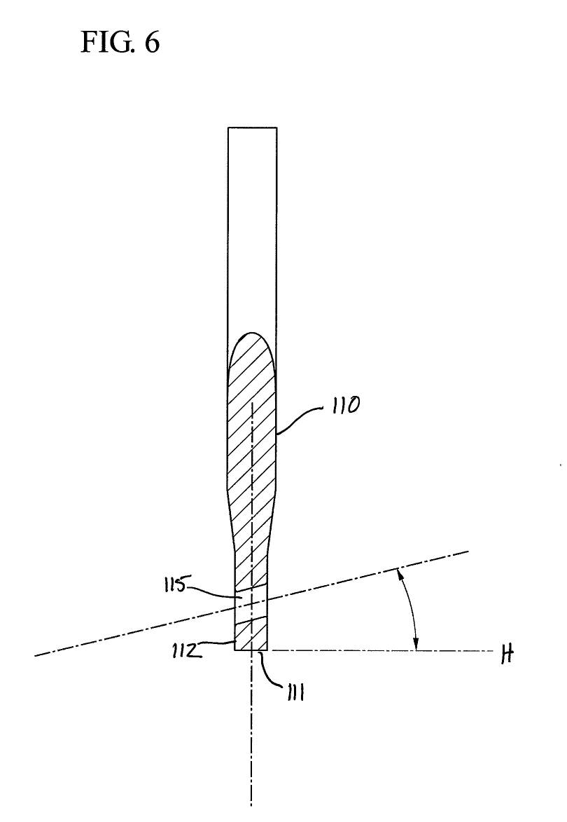

Each of left link 100 and right link 200 includes an attachment portion 110 and 210 respectively that is adapted to be attached to the sacrum. Left link 100 has a first end defining a left link attachment flange 111, which in turn defines a left link attachment opening 115. Right link 200 has a first end defining a right link attachment flange 211, which in turn defines a right link attachment opening 215. Left link attachment flange 111 has a face 112 defining a plane that may be generally aligned with or parallel to a plane defined by left link 100. Similarly, right link attachment flange 211 has a face 212 defining a plane that may be generally aligned with or parallel to a plane define by right link 200. Left link attachment opening 115 and right link attachment opening 215 are configured to allow a fixation device 300 to extend therethrough. Each of left link attachment opening 115 and right link attachment opening 215 are angled in two directions. As seen in FIG. 5, each of left link attachment opening 115 and right link attachment opening is oriented between about 45 degrees and 60 degrees toward the longitudinal plane LP of flexing link assembly 10. In other words. As shown in FIG. 5, left link attachment opening 115 is oriented such that a longitudinal plane extending through left link attachment opening 115 intersects the longitudinal axis of flexing link assembly 10 at an angle a of between about 45 degrees and about 60 degrees. And, as shown in FIG. 5, right link attachment opening 215 is oriented such that a longitudinal axis extending through right link attachment opening 215 intersects the longitudinal plane of flexing link assembly 10 at an angle β of between about 45 degrees and about 60 degrees. As shown in FIG. 6, left link attachment opening 115 is oriented so it is between about 5 degrees and 10 degrees above the horizontal line H. Although not shown, right link attachment opening 215 has this same orientation. This “compound” angle for both left link attachment opening 115 and right link attachment opening 215 allows attachment portions 110 and 210 to fit more securely on the surface of the sacrum.

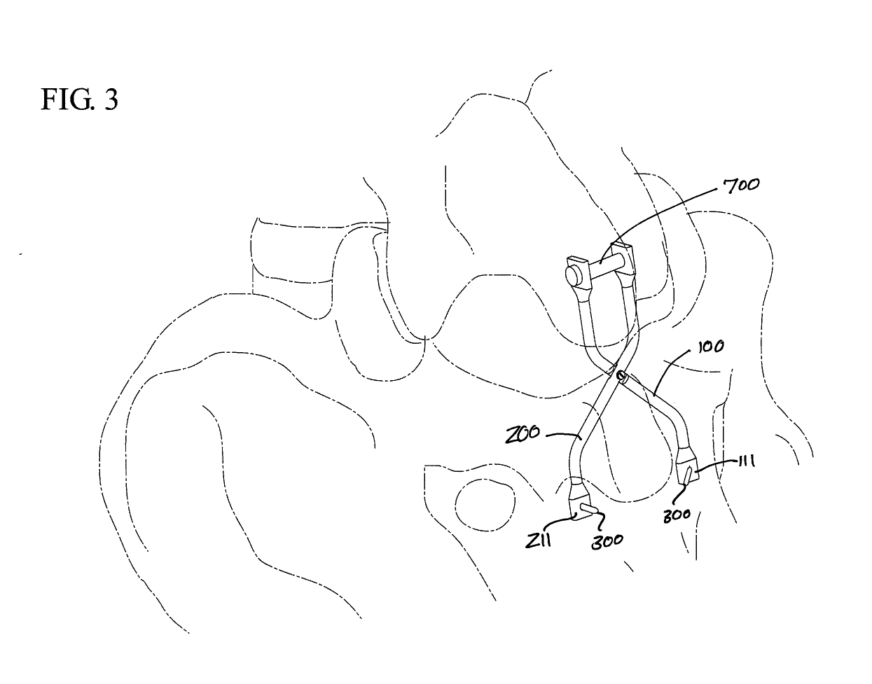

Any fixation device 300 may be used to attach each link 100, 200 to the sacrum. For example, a standard facet screw may be used for this purpose, with a separate screw extending through left link attachment opening 115 and right link attachment opening 215. Each of left link 100 and right link 200 may be implanted in the patient so that their respective attachment portions 110, 210 are located on opposite sides of the medial sacral crest. In addition, left link attachment opening 115 should be oriented so that fixation device 300 for left link attachment flange 111 can be located in the right pedicle. Similarly, right link attachment opening 215 should be oriented so that fixation device 300 for right link attachment flange 211 can be located in the left pedicle. See e.g. FIG. 3. This portion of the sacrum lies in an area away from the cauda equina nerve roots and is a relatively safe area in which to affix flexing link assembly 10. In addition, the pedicles of the sacrum provide enough support to allow flexing link assembly 10 to provide distraction between the L5 and S1 vertebrae. Left link attachment portion 110 extends generally vertically from left link attachment opening 115. Similarly, right link attachment portion 210 extends generally vertically from right link attachment opening 215.

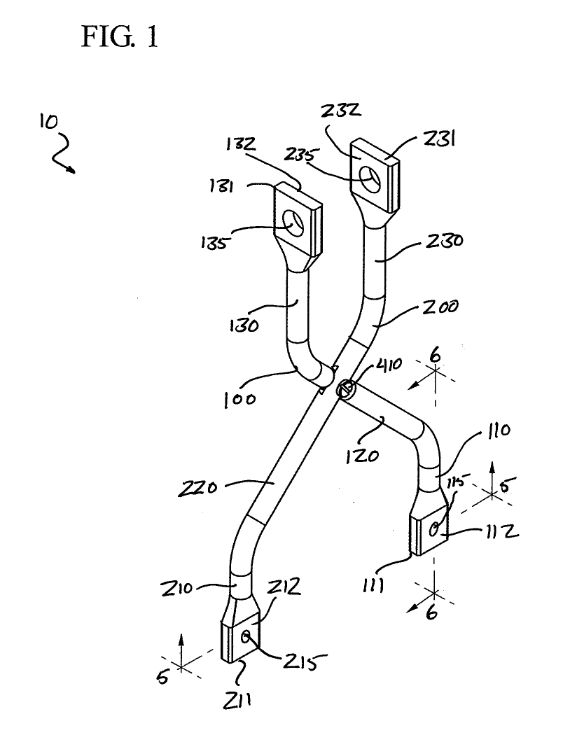

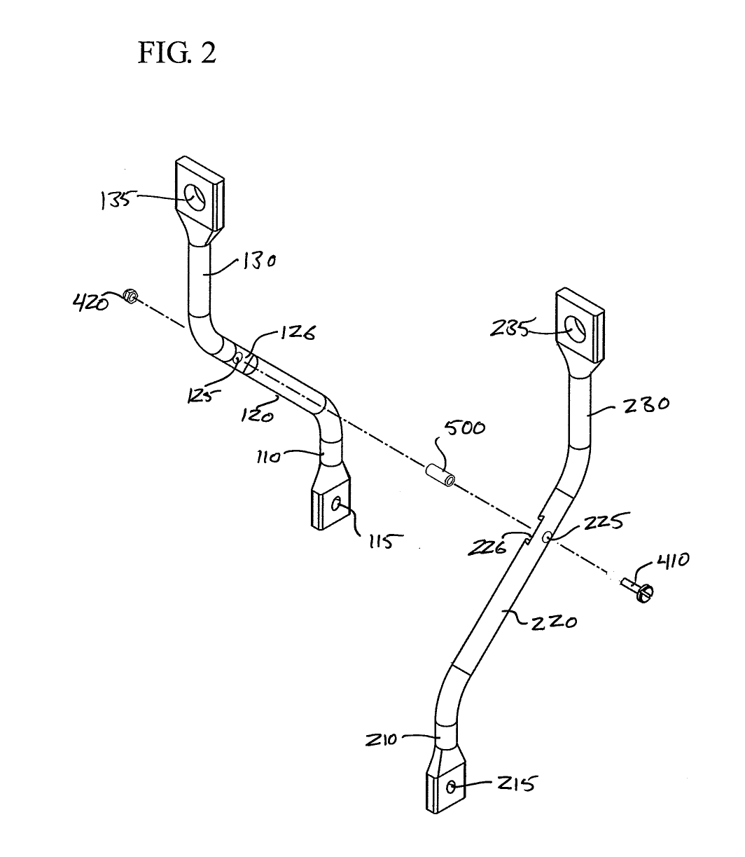

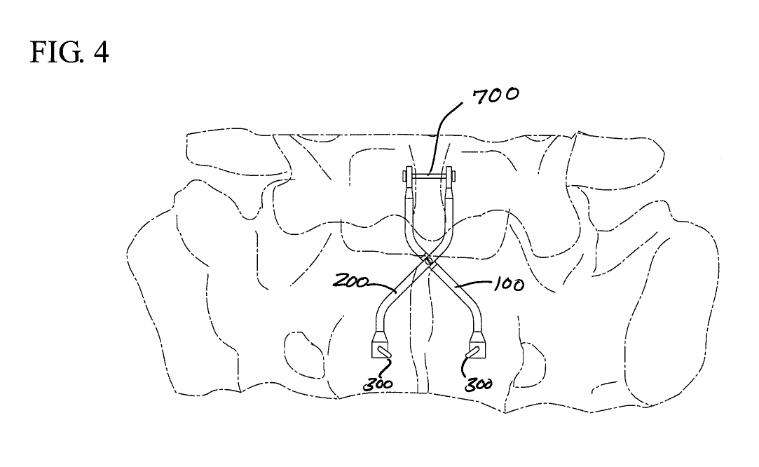

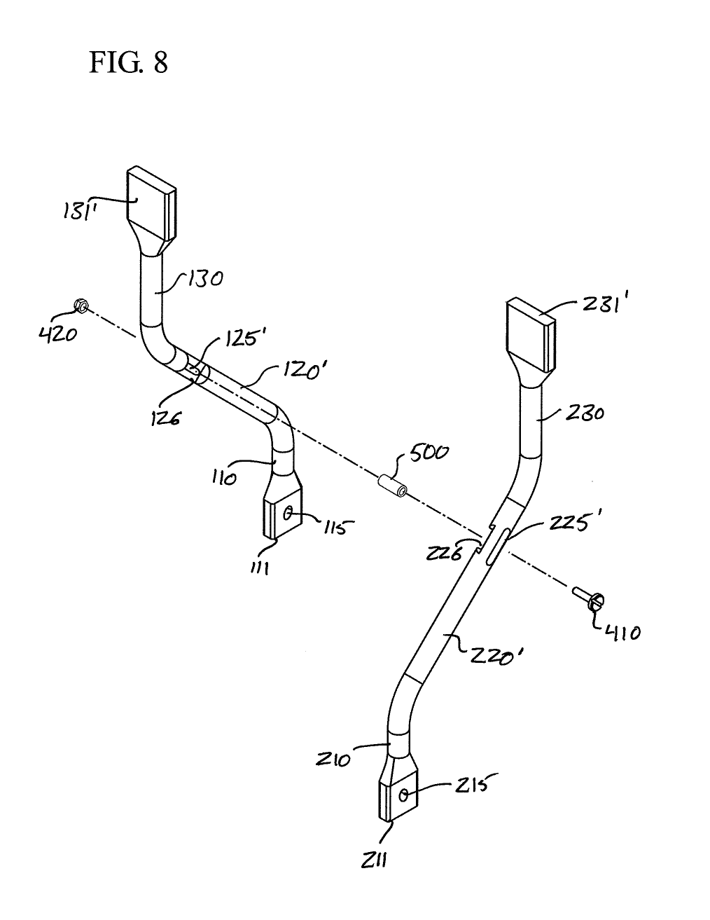

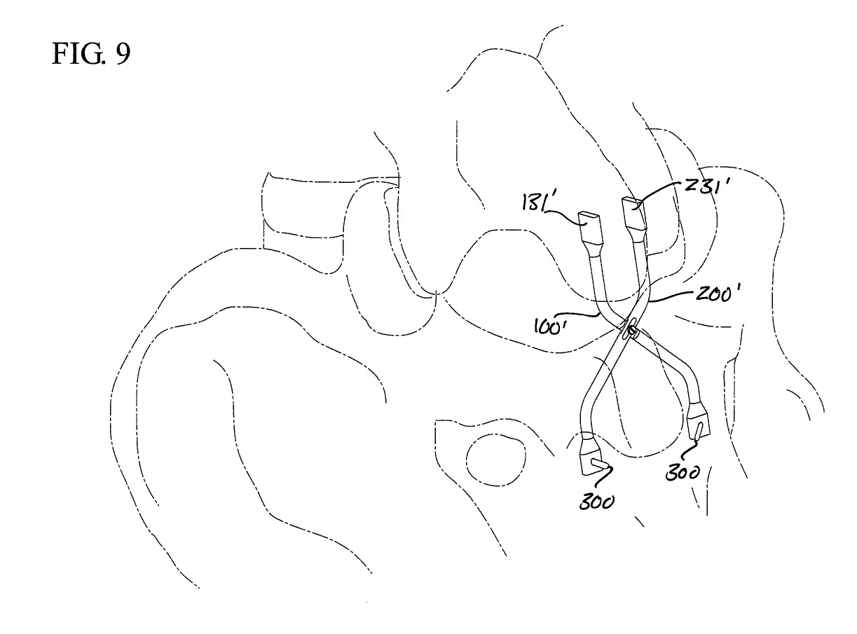

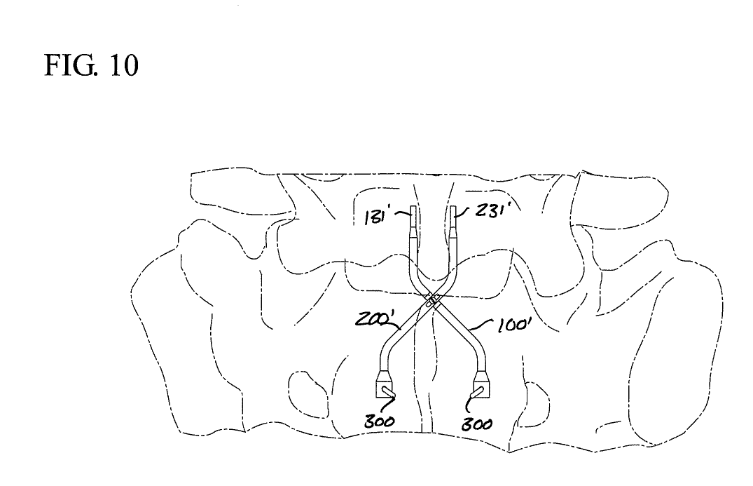

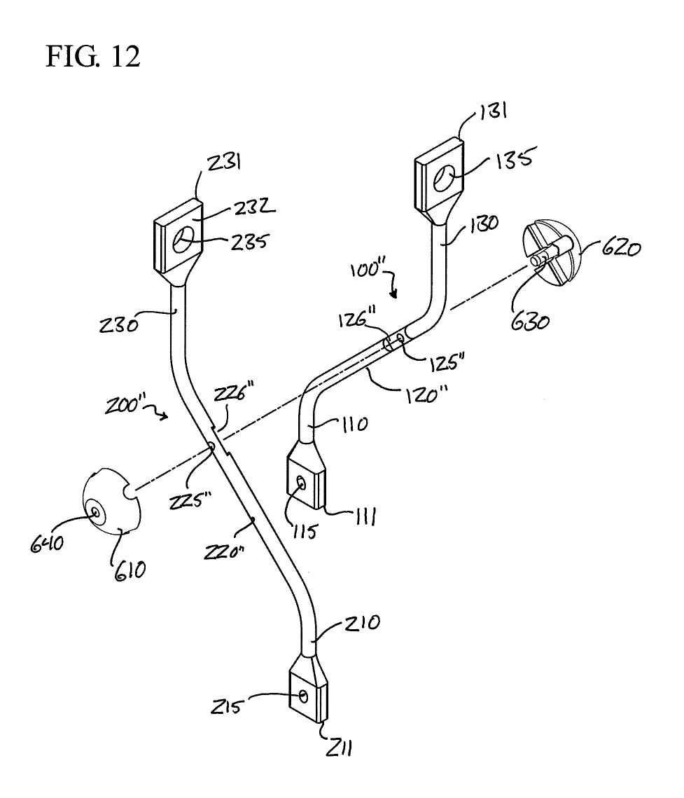

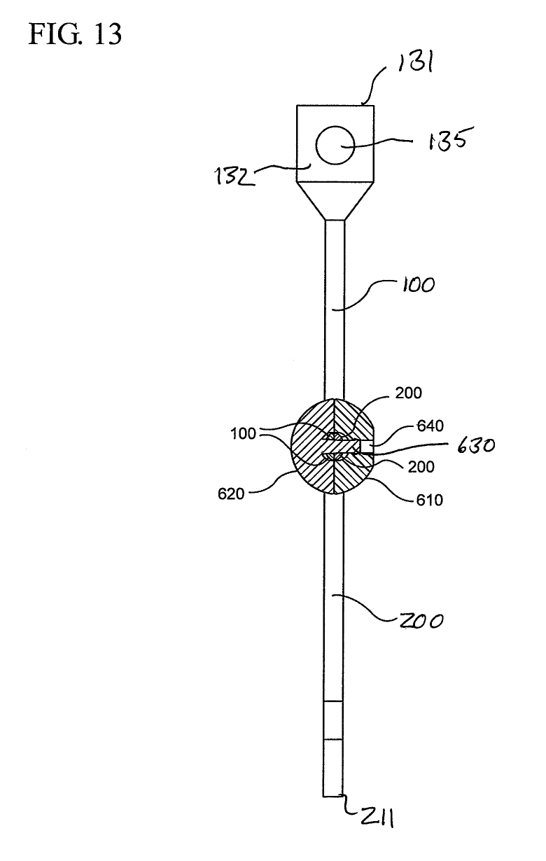

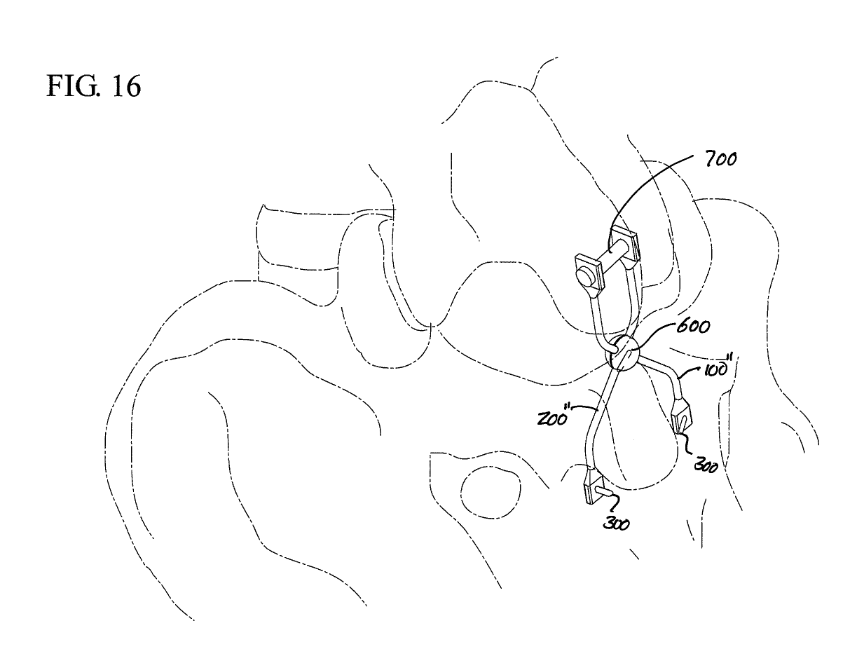

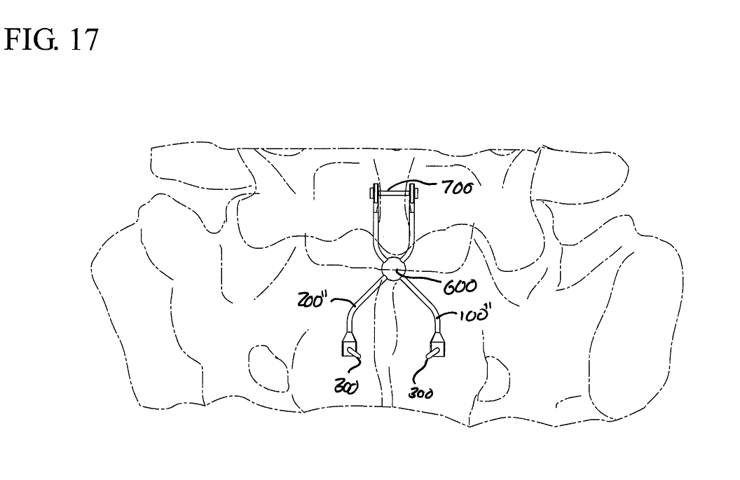

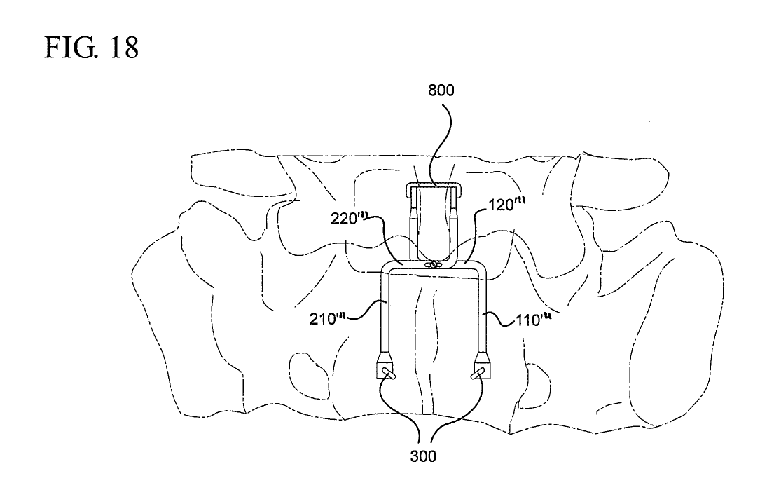

Each of left link 100 and right link 200 includes an intermediate portion 120 and 220 respectively extending from left and right attachment portion 110 and 210 respectively. In one embodiment, left link intermediate portion 120 extends from a second end of left link attachment portion 120 in a generally lateral and superior direction. See FIGS. 1-13 and FIGS. 16 and 17. Alternatively, left link intermediate portion 120″′ may extend from a second end of left link attachment portion 110″′ in a generally lateral direction, i.e. left link intermediate portion 120″′ extends substantially perpendicular to left link attachment portion 110″′. See FIG. 18. Right link intermediate portion 220 is substantially the same as left link intermediate portion 120, but it is rotated 180 degrees about its longitudinal axis when flexing link assembly 10 is assembled. Thus, right link intermediate portion 220 may extend from a second end of right link attachment portion 210 in a generally lateral and superior direction. See FIGS. 1-13 and FIGS. 16 and 17. Alternatively, right link intermediate portion 220″′ may extend from a second end of right link attachment portion 210″′ in a generally lateral direction, i.e. right link intermediate portion 220″′ extends substantially perpendicular to right link attachment portion 210″′. See FIG. 18. The particular angle at which left link intermediate portion 120 extends from left link attachment portion 110 and at which right link intermediate portion 220 extends from right link attachment portion 210 can vary depending on the particular patient anatomy and the particular location in the spine where flexing link assembly 10 is to be located. It is also possible for left link intermediate portion 120 to have a different configuration than right link intermediate portion 220. However, left link intermediate portion 120 and right link intermediate portion 220 should cross somewhere adjacent to the midline of the spine, i.e. along the sagittal plane, onto which flexing link assembly 10 is to be implanted. This will allow left link 100 and right link 200 to be connected together such that the connection is adjacent to the sagittal plane of the patient. This orientation provides equal loading on flexing link assembly 10 during extension of the spine.

Left link intermediate portion 120 defines a left link intermediate opening 125. Similarly, right link intermediate portion 220 defines a right link intermediate opening 225. Left link intermediate opening 125 and right link intermediate opening 225 are arranged on left link intermediate portion 120 and right link intermediate portion 220 respectively such that when they cross each other left link intermediate opening 125 and right link intermediate opening 225 are aligned. This allows an attachment mechanism 400 to connect left link 100 and right link 200. A notch 126 can be formed around left link intermediate opening 125 and a notch 226 can be formed around right link intermediate opening 226 to facilitate the connection of left link 100 and right link 200 together. The specific configuration of each notch should allow ease of engagement between each link and also allow relative movement between the links. This can be facilitated by forming each notch with rounded edges. Attachment mechanism 400 may include a connecting pin 410 and a locking nut 420. Alternatively, a self-locking screw, a locking tongue and groove arrangement formed on left intermediate portion 120 and right intermediate portion 220 or any other locking mechanism that will connect the links together but still allow relative motion therebetween, may be used. A damper sleeve 500 may be located around the shaft of the connecting pin 410 forming attachment mechanism 400. Damper sleeve 500 may be formed of silicone and may have a durometer of between about 63 A and 80 A. Damper sleeve 500 thus allows some movement between left link 100 and right link 200.

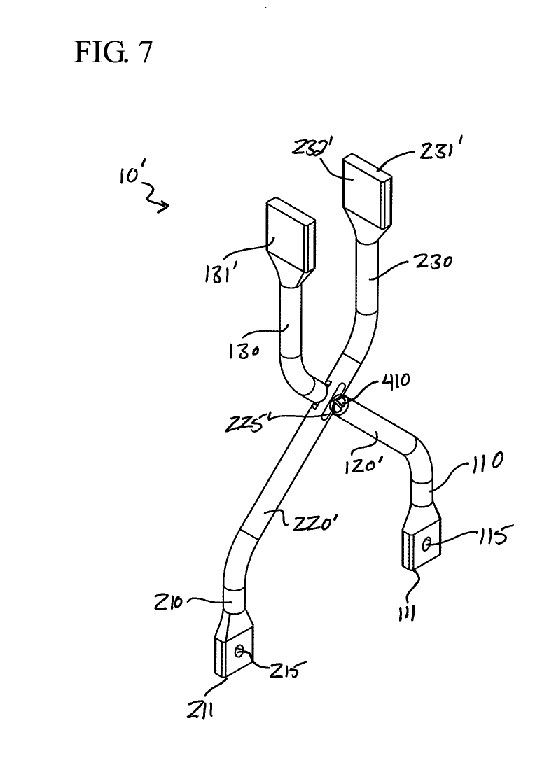

Left link intermediate opening 125′ and right link intermediate opening 225′ may also be dimensioned so that attachment mechanism 400 may travel within left link intermediate opening 125′ and right link intermediate opening 225′ to provide a slip-lock type of connection between left link 100′ and right link 200′. For example, as shown in FIGS. 7-10, left link intermediate opening 125′ and right link intermediate opening 225′ are elongated slots. This allows attachment mechanism 400 to travel along the length of such slots, and thus one of the links may move with respect to the other of the links, when the spine is in flexion or when the spine laterally bends or rotates. However, the orientation of the slots limits the travel of attachment mechanism 400, and thus the links are limited in their ability to move with respect to one another, when the spine is in extension. In addition, damper sleeve 500 may still be used in combination with flexing link assembly 10′ of this embodiment to allow additional flexibility and motion between left link 100′ and right link 200′ when they are connected together.

If desired, the surfaces of links 100 and 200 adjacent to the location where they are connected together may be ground in a manner that facilitates mating and engagement between the two surfaces. For example, complementary grooved configurations may be etched into the surface of left link intermediate portion 120 and the surface of right link intermediate portion 220 that contact each other when left link 100 is connected to right link 200.

Each of left link 100 and right link 200 also includes a mounting portion 130 and 230 respectively extending from intermediate portion 120 and 220 respectively and adapted to facilitate engagement with the spinous process of the L5 vertebra. Both left mounting portion 130 and right mounting portion 230 extend in a generally superior direction from left link intermediate portion 120 and right link intermediate portion 220 respectively. The foregoing described configuration of each link 100 and 200 allows mounting portions 130 and 230 to be located in close proximity to the spinous process of the L5 vertebra. More specifically, the configuration of each link 100 and 200 positions each mounting portion 130 and 230 on the opposite side of the sagittal plane from where attachment portions 110 and 210 respectively are attached to the sacrum. In other words, left mounting portion 130 is located adjacent to the left side of the spinous process of the L5 vertebra while left link attachment portion 110 is located adjacent to the right pedicle of the sacrum and right mounting portion 230 is located adjacent to the right side of the spinous process of the L5 vertebra while right link attachment portion 210 is located adjacent to the left pedicle of the sacrum.

Left mounting portion 130 defines a left mounting flange 131, which in turn defines a left mounting opening 135. Right mounting portion 230 defines a right mounting flange 231, which in turn defines a right mounting opening 235. Left mounting flange 131 has a face 132 defining a plane that may be generally orthogonal to the plane defined by left link 100. Similarly, right mounting flange 231 has a face 232 defining a plane that may be generally orthogonal to the plane defined by right link 200. Mounting portions 130 and 230 can thus be readily attached to the L5 spinous process so as to maintain decompression between the S1 and the L5 vertebrae. Any suitable fixation mechanism can be used for this purpose, such as screws 700 or spikes that extend through left mounting opening 135 and right mounting opening 235. In addition, tethers 800 can be used. Alternatively, mounting portions 130 and 230 do not have to be attached to the spinous process of the L5 vertebra. Instead they may together form a cupped configuration that engages the inferior surface of the spinous process of the L5 vertebra to contact an inferior surface of the spinous process of the L5 vertebra to maintain decompression. See e.g. FIGS. 7-10. In this embodiment left mounting flange 131′ does not define an opening therein. Similarly, right mounting flange 231′ also does not define an opening therein.

The portion of flexing link assembly 10 that engages the inferior surface of the spinous process of the L5 vertebra is defined by left link intermediate portion 120 and right link intermediate portion 220 around the location where attachment mechanism 400 connects left link 100 to right link 200. Moving this location closer to or farther from left mounting portion 130 and right mounting portion 230 varies the amount of distraction that can be realized from flexing link assembly 10.

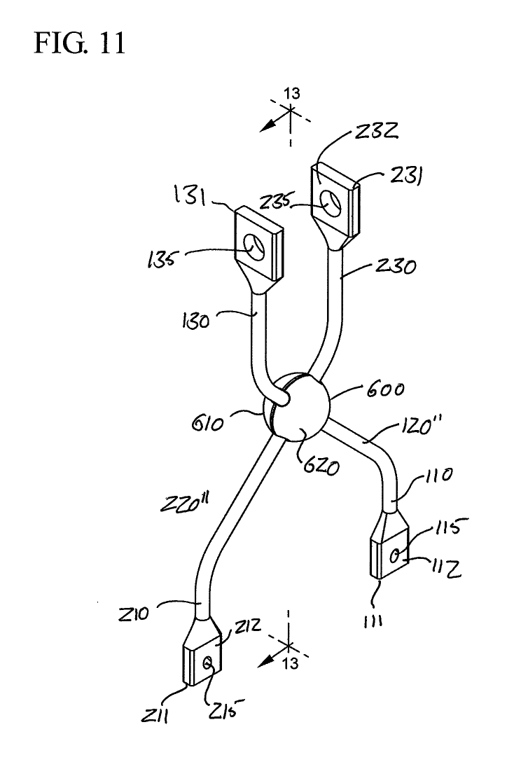

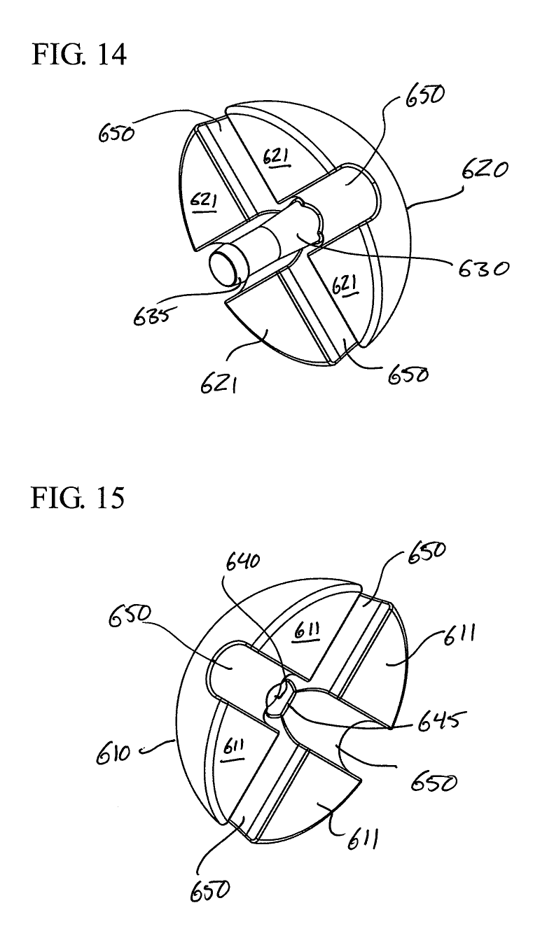

In another embodiment of the flexing link assembly, an attachment device 600 such as illustrated in FIGS. 11-17 may be used to connect left link 100 and right link 200. Attachment device 600 is formed from a flexible material similar to the material used to form damper sleeve 500. Thus, silicone having a durometer of between about 63 A and 80 A may be used to form ball joint 600. This allows relative movement between left link 100″ and right link 200″ when they are connected via attachment device 600. If desired, portions of attachment device 600 may be formed from a harder material, as will be described in more detail hereinafter.

Attachment device 600 may have a generally spherical outer configuration, although the specific outer configuration for attachment device 600 can take any shape. Attachment device 600 is formed from two halves, a first half 610 and a second half 620. First half 610 defines a first generally planar face 611 and second half 620 defines a second generally planar face 621. Each face 611 and 621 are placed adjacent to each other to connect each half and links 100″ and 200″ together. First half 610 and second half 620 are connected together through the use of a locking stem 630 formed on one half of attachment device 600 that engages with and is coupled to a locking recess 640 formed on another half of attachment device 600. Either half can include locking stem 630, but in the embodiment illustrated in the FIGS. 11-17, second half 620 includes locking stem 630 while first half 610 defines locking recess 640. Locking stem 630 may be formed as a separate piece. Locking stem 630 and locking recess 640 include compatible detents to allow locking stem 630 and locking recess 640 to be locked together when locking stem 630 is pushed into locking recess 640. For example, the end of locking stem 630 may include a flange 635 while locking recess 640 may include an undercut 645 that engages with the underside of flange 635 to hold locking stem 630 in place once flange 635 is pushed past undercut 645. Locking stem 630 is dimensioned so that it fits through left link intermediate opening 125″ and right link intermediate opening 225″. Channels 650 are formed in each face 611 and 621 so left link intermediate portion 120″ and right link intermediate portion 220″ can be nested therein. Channels 650 may have a flared or straight configuration where they exit first half 610 and second half 620. The angle at which the channels cross each other is determined by the particular orientation that left link intermediate portion 120″ and right link intermediate portion 220″ cross each other when flexing link assembly 10″ is implanted and fixed to the sacrum. Because attachment device 600, including locking stem 630, is made from a flexible material such as silicone, left link 100″ and right link 200″ are able to flex or move with respect to one another even when they are connected together through the use of attachment device 600. If desired, locking stem 630 may be formed from a harder material than the rest of attachment device 600. For example, locking stem 630 could be formed from PEEK, steel, titanium and still allow flexing link assembly to flex.

The embodiment of the flexing link assembly shown in FIGS. 11-17 also shows notches 126″ and 226″ formed on the other side of left link 100″ and right link 200″ respectively than in the previous embodiments. This allows left link 100″ to be located posterior of right link 200″. In the other embodiments, left link 100 is located anterior to right link 200. It does not matter which link is posterior and which link is anterior. Either link may be posterior in each of the embodiments of the flexing link assembly described herein.

An example of a method to secure flexing link assembly 10 within a spinal column will now be described. Once the patient has been sedated and the entry point has been determined, a midline incision may be made over the spinous process exposing the supraspinous ligament overlying the spinous processes at the symptomatic level(s). The supraspinous ligament is preserved. Alternatively, if the supraspinous ligament is compromised, it is not necessary for the surgeon to preserve the supraspinous ligament. The interspinous ligament is dilated and the interspinous space is distracted. One link, either left link 100 or right link 200 may then be inserted through the interspinous ligament. Where left link 100 is inserted first, it is inserted such that left mounting portion 130 is positioned adjacent to the left of the spinous process of the vertebra of interest, typically the L5 vertebra. If right link 200 is inserted first, it is inserted such that right mounting portion 230 is positioned adjacent to the right of the desired spinous process. If desired, the mounting portion can be affixed to the desired spinous process. This process is repeated with the other link. Instead of having each mounting portion affixed to the desired spinous process individually after each link is inserted, the mounting portions of the two links can be both affixed to the desired spinous process after both links have been inserted and appropriately positioned. With left link 100 and right link 200 aligned such that left link intermediate opening 125 and right link intermediate opening 225 are aligned, attachment mechanism 400 can be connected through the two intermediate openings 125 and 225 to movably connect left link 100 and right link 200. Thereafter, fixation devices, such as facet screws, may be inserted through left link attachment opening 115 and right link attachment opening 215 to fix left link 100 and right link 200 respectively to the desired vertebra, typically the sacrum. As described above, left link attachment opening 115 is located adjacent to the right pedicle of the sacrum and right link attachment opening 215 is located adjacent to the left pedicle of the sacrum. The interspinous ligament and the L5 spinous process and the S1 spinous process may need to be dissected in order to accommodate attachment mechanism 600. In addition, the surgeon may have to remove any bony structures, such as spurs, on the sacrum and the L5 spinous process that could interfere with the fixation of flexing link assembly 10 to those portions of the spine.

Alternatively, flexing link assembly 10 may be inserted in one piece with left link 100 and right link 200 connected prior to and during the insertion procedure. In addition, attachment mechanism 400 may be adjusted to lock left link 100 to right link 200 during the insertion process. Attachment mechanism 400 can thereafter be “loosened” to allow flexing, i.e. relative movement between left link 100 and right link 200.

The flexing link assembly described herein can be constructed with various biocompatible materials such as, for example, titanium, titanium alloy, surgical steel, biocompatible metal alloys, stainless steel, Nitinol, plastic, polyetheretherketone (PEEK), carbon fiber, ultra-high molecular weight (UHMW) polyethylene, and other biocompatible polymeric materials. In addition, left link 100 and right link 200 may have any cross-sectional shape, such as, but not limited to, circular, elliptical, polygonal, and dog-bone shaped. Furthermore, left link 100 may have a different cross section than right link 200.

While various embodiments of the flexing link assembly have been described above, it should be understood that they have been presented by way of example only, and not limitation. Many modifications and variations will be apparent to the practitioner skilled in the art. The foregoing description of the flexing link assembly is not intended to be exhaustive or to limit the scope of the invention. It is intended that the scope of the invention be defined by the following claims and their equivalents.

What is claimed is:

1. An apparatus, comprising:
a first link having an attachment portion adapted to be coupled to a first vertebra of a spinal column, an intermediate portion connected to the attachment portion and a mounting portion connected to the attachment portion and adapted to facilitate engagement with a spinous process of a second vertebra of the spinal column;
a second link having an attachment portion adapted to be coupled to the first vertebra, an intermediate portion connected to the attachment portion and a mounting portion connected to the intermediate portion and adapted to facilitate engagement with the spinous process of the second vertebra; and
a connector connecting the first link to the second link and allowing relative movement between the first link and the second link.

2. The apparatus of claim 1 wherein the attachment portion and the mounting portion of the first link are substantially parallel to each other and the intermediate portion of the first link extends laterally between the attachment portion and the mounting portion.

3. The apparatus of claim 2 wherein the attachment portion and the mounting portion of the second link are substantially parallel to each other and the intermediate portion of the second link extends laterally between the attachment portion and the mounting portion.

4. The apparatus of claim 2 wherein the attachment portion and the mounting portion of the first link are connected to the intermediate portion of the first link at a non-orthogonal angle.

5. The apparatus of claim 3 wherein the attachment portion and the mounting portion of the second link are connected to the intermediate portion of the second link at a non-orthogonal angle.

6. The apparatus of claim 1 wherein the intermediate portion of the first link defines a first opening therein and the intermediate portion of the second link defines a second opening therein and the connector extends through the first opening and the second opening.

7. The apparatus of claim 6 further comprising a resilient sleeve extending through the first opening and the second opening and around the connector.

8. The apparatus of claim 6 wherein the first opening defines a first major axis and the second opening defines a second major axis and the connecter defines a third major axis, and wherein the first major axis and the second major axis are greater than the third major axis.

9. The apparatus of claim 1 wherein when the connecter is located adjacent to a longitudinal axis of the apparatus.

10. The apparatus of claim 1 wherein the attachment portion of the first link defines a first attachment opening and the attachment portion of the second link defines a second attachment opening and wherein the first attachment opening and the second attachment opening each defines a longitudinal axis that extends between about 5 degrees and about 10 degrees above a plane orthogonal to a plane defined by the first attachment opening, the second attachment opening and the connector.

11. The apparatus of claim 1 wherein the attachment portion of the first link defines a first attachment opening and the attachment portion of the second link defines a second attachment opening and wherein the first attachment opening and the second attachment opening each defines a longitudinal axis that extends between about 45 degrees and about 60 degrees toward a longitudinal axis of the apparatus.

12. The apparatus of claim 10 wherein the longitudinal axis of the first attachment opening and the longitudinal axis of the second attachment opening each extends between about 45 degrees and about 60 degrees toward a longitudinal axis of the apparatus.

13. An apparatus, comprising:
a first link having a first attachment portion defining a first opening therein to allow a first fastening mechanism to extend therethrough to couple the first link to a first vertebra of a spinal column, a first intermediate portion extending laterally and distally from the first attachment portion, and a first mounting portion extending from the first intermediate portion and extending distally from the first attachment portion wherein the first mounting portion is adapted to facilitate engagement with a first side of a spinous process of a second vertebra of the spinal column and the first intermediate portion defines a first intermediate opening therein;
a second link having a second attachment portion defining a second opening therein to allow a second fastening mechanism to extend therethrough to couple the second link to the first vertebra, a second intermediate portion extending laterally and distally from the second attachment portion, and a second mounting portion extending from the second intermediate portion and extending distally from the second attachment portion wherein the second mounting portion is adapted to facilitate engagement with the spinous process of the second vertebra on a second side of the spinous process of the second vertebra and the second intermediate portion defines a second intermediate opening therein; and
a connector extending through the first intermediate opening and the second intermediate opening such that the connector connects the first link to the second link and the first link is movable with respect to the second link.

14. The apparatus of claim 13 wherein the first intermediate opening has a generally elongate shape and defines a first major axis and the second intermediate opening has a generally elongate shape and defines a second major axis and the connecter defines a diameter and wherein the first major axis and the second major axis are greater than the diameter.

15. The apparatus of claim 13 wherein when the connecter is located adjacent to a longitudinal axis of the apparatus.

16. The apparatus of claim 13 further comprising a damper surrounding a portion of the connector and disposed in the first intermediate opening and the second intermediate opening.

17. The apparatus of claim 13 wherein the first intermediate portion includes a first surface texture and the second intermediate portion includes a second surface texture complementary to and in contact with the first surface texture.

18. The apparatus of claim 13 wherein the attachment portion of the first link defines a first attachment opening and the attachment portion of the second link defines a second attachment opening and wherein the first attachment opening and the second attachment opening each defines a center line that extends between about 5 degrees and about 10 degrees above a plane orthogonal to a plane defined by the first attachment opening, the second attachment opening and the connector and wherein the centerline of the first attachment opening and the second attachment opening each defines a center line that extends between about 45 degrees and about 60 degrees toward a longitudinal axis of the apparatus.

19. A method of implanting a device having a first link, a second link and a connector, comprising:
engaging a portion of the first link with a first side of a spinous process of a first vertebra of a spinal column along a first side of a sagittal plane;
engaging a portion of the second link with a second side of the spinous process of the first vertebra along a second side of the sagittal plane;
attaching a portion of the first link to a second vertebra of a spinal column along the second side of the sagittal plane; and
attaching a portion of the second link to the second vertebra along the first side of the sagittal plane.

20. The method of claim 19 further comprising aligning the connector along the sagittal plane.
